(19) United States
(12) Patent Application Publication
(10) Pub. No.: US 2015/0150829 A1
(43) Pub. Date: Jun. 4, 2015

(54) CAPSULES CONTAINING THYMOQUINONE

(71) Applicant: DSM IP ASSETS B.V., Heerlen (NL)

(72) Inventors: Stephane Etheve, Basel (CH); Kevin Prudence, Basel (CH); Loni Schweikert, Basel (CH); Aniko Szepes, Basel (CH)

(21) Appl. No.: 14/412,222

(22) PCT Filed: Jun. 24, 2013
(86) PCT No.: PCT/IB2013/055167

§ 371 Date: Dec. 31, 2014

Related U.S. Application Data
(60) Provisional application No. 61/666,988, filed on Jul. 2, 2012.

(51) Int. Cl. A61K 31/122; A61K 31/05; A61K 36/53; A61K 9/48

(52) U.S. Cl. CPC A61K 31/122; A61K 9/4825; A61K 31/05; A61K 36/53; A61K 9/4816; A61K 9/4858

(57) ABSTRACT

This invention relates to stable capsule formulations for pharmaceutical, nutraceuticals or food supplements which contain thymoquinone (THQ) as an active ingredient, and which can be stored at room temperature without significant loss of THQ during the shelf life of the capsules. The preferred capsules have a hydroxypropylmethylcellulose (HPMC) shell and may contain carvacrol as an additional active ingredient, either in synthetic form, or as part of a plant extract. Optionally, the capsules may contain a viscosity agent.

### BRIEF DESCRIPTION OF THE INVENTION

This invention relates to stable capsule formulations for pharmaceutical, nutraceuticals or food supplements which contain thymoquinone (THQ) as an active ingredient, preferably as an ingredient in an oregano extract, and which can be stored at room temperature without significant loss of THQ during the shelf life of the capsules. The preferred capsules have a hydroxypropylmethylcellulose (HPMC) shell and may contain carvacrol as an additional active ingredient, either in synthetic form, or as part of a plant extract. Optionally, the capsules may contain a viscosity agent.

### BACKGROUND OF THE INVENTION

Capsules are constructed with hard or soft shells and contain a single dose of one or more active ingredients. They are mainly intended for oral administration and act as containers for powders, pellets or microtablets. They also enable transformation of a liquid or semi-solid formulation into a solid single-unit dosage form with improved content uniformity and accurate dosage. The capsule shell is suitable for taste or odour masking. Furthermore, it can improve stability of the fill via providing protection against oxygen and light. Capsules are available in various shapes and sizes. They possess a smooth surface, which ensures a comfortable and convenient administration.

The US and European Pharmacopoeias distinguish between hard, soft and modified-release capsules.

Hard capsules have two prefabricated cylindrical sections (body and cap) that fit together. One end of each section is rounded and closed, and the other is open. Hard capsules are usually filled with solid substances (e.g. powder or granules), but in smaller scale productions, liquids or semi-solids may be encapsulated.

Soft capsules usually contain a liquid or semi-solid fill and are usually oblong or oval in shape. They are formed, filled and sealed in one operation via the rotary die process. This technology generally requires large amount of material. Therefore, soft capsule manufacturing is difficult to conduct at the laboratory scale.

Gelatin is a tasteless, odourless and water-soluble substance that undergoes a reversible phase change from a solution to a gel governed by temperature. These characteristics promoted its utilization as raw material for hard capsules. However gelatin has a few drawbacks which limits its use in this application: its hygroscopicity, the relatively high water content of gelatin capsules (13-16%) and the tendency to cross-linking. Furthermore, gelatin can be problematic because of religious, cultural or dietary considerations (vegetarians, patients with restricted diets or healthy life-style) and because of the risk of transmitting spongiform encephalopathy (BSE).

Gelatin can often be replaced by hydroxypropylmethylcellulose (HPMC) or pullulan to make animal-free or all-vegetable capsules. HPMC is a plant-derived, odourless and tasteless polymer which is considered to be resistant to cross-linking. Since HPMC capsules possess low moisture content (2-5%), they are more suitable for moisture sensitive formulations than gelatin. However, interactions of HPMC coatings with antioxidant polyphenolic actives (present e.g. in plant extract such as green tea) have been observed.

Pullulan is a natural water-soluble polysaccharide, produced via starch fermentation. The low oxygen permeability of pullulan films enhances protection of encapsulated oxidation-sensitive ingredients.

The benefits of oregano extracts have been described, e.g. in WO 08/017484 (DSM IP Assets, B.V). Oregano extract contains a number active ingredients, including carvacrol (CRV) and thymoquinone (THQ). While carvacrol is a relatively stable molecule in various formulations, THQ is not, and when oregano extract is formulated conventionally the THQ degrades quickly.

WO 09/150179 (DSM IP Assets, B.V.) uses polyunsaturated fatty acids (PUFAs) and their ethyl esters (PUFA EEs) to stabilize oregano extract for use in various capsules. The PUFAs/PUFA EEs prevent a waxy precipitation of the extract, and thus stabilize the extract as a whole, but the PUFAs do not protect the THQ from degradation.

WO 10/094761 (DSM IP ASSETS, B.V.) describes a capsule (LiCaps) containing oregano extract, tricylglycerol (DURKEX 200), and phosphatidylcholine. These capsules were stored at 4° C. to prevent degradation of the THQ, and thus are not suitable for storage at room temperature.

There are numerous examples of commercially available oregano extract capsules. However, these do not contain a substantial amount of THQ, and thus the recipients do not receive the full benefit of the oregano extract. It would be desirable to have a capsule which preserves the THQ present in its fill, and can be stored at room temperature.

### DETAILED DESCRIPTION OF THE INVENTION

It has been found, in accordance with this invention, that the stability of THQ in can be maintained at room temperature in a nutraceutical or pharmaceutical capsule comprising:
a) shell selected from the group consisting of: hydroxypropylmethylcellulose (HPMC), pullulan, and gelatin;
b) a fill composition comprising at least 2 weight % thymoquinone (THQ), preferably from 2-8 weight % thymoquinone;
c) a diluent selected from the group consisting of: middle chain triglycerides, glycerol, edible vegetable waxes; plant oils (for example: olive oil, palm oils, sunflower oils, maize/corn oil, soybean oil, sesame oil, or rice bran oil), and mixtures thereof; and
d) a viscosity increasing agent, with the proviso that the viscosity increasing agent is not phosphaditylcholine; and with the further proviso that when the shell is pullulan or gelatin, then the viscosity increasing agent comprises neither phosphatidylcholine/lecithin.

In preferred embodiments, the THQ is present in combination with other active ingredients, such as carvacrol (CRV). These ingredients may be present in a plant extract, particularly in an oregano extract.

One important aspect of this invention is that the capsules can be stored at room temperature over the shelf life of the capsule and the THQ is still present. In previous oregano extract capsules stored at room temperature, the THQ is quickly degraded, and if it is present at all in the capsule, it is present only at a significantly reduced amount (i.e. less than 1% by weight).

In accordance with this invention, the THQ is present at an amount of at least 2% by weight for the shelf life of the capsule. The “shelf life” of a nutraceutical is typically two years from the date of manufacture, and is generally indicated on the packaging. Thus, in the present invention, the capsule contains oregano extract wherein the THQ is present in an amount of at least 2% by weight for at least two years after the date of manufacture when stored at room temperature.

### BRIEF DESCRIPTION OF THE FIGURES

FIG. 1 is a set of graphs showing the stability of THQ in gelatin capsules during storage.

FIG. 1a is at 25° C./60% relative humidity (RH) and FIG. 1b is at 40° C./75% RH.

FIG. 2 is a set of graphs showing the stability of CRV in gelatin capsules during storage. (FIG. 2a is at 25° C./60% RH and FIG. 2b is at 40° C./75% RH).

FIG. 3 is a set of graphs showing stability of THQ in HPMC capsules during storage (FIG. 3a is at 25° C./60% RH and FIG. 3b at 40° C./75% RH)

FIG. 4 is a set of graphs showing the stability of CRV in HPMC capsules during storage (FIG. 4a is at 25° C./60% RH & and FIG. 4b is at 40° C./75% RH)

FIG. 5 is a set of graphs showing the stability of THQ in pullulan capsules during storage (FIG. 5a is at 25° C./60% RH and FIG. 5b is at 40° C./75% RH)

FIG. 6 is a set of graphs showing the stability of CRV in pullulan capsules during storage (FIG. 6a is at 25° C./60% RH & FIG. 6b is at 40° C./75% RH)

### Oregano Extract Containing THQ

If a plant extract is the source of THQ, then it is preferred that it is an oregano extract. The “oregano extracts” may be of any origin from a plant (whole plant or parts thereof) belonging to the genera Origanum such as Origanum vulgare or O. minutiflores and Thymus such as Thymus vulgaris in form of a concentrate of extractable compounds, especially volatile compounds. Further examples of plants from the genus Origanum covered by the term “oregano”, are O. majorana, O. dictamus, O. creticum, O.x majoricum, O. aureum, O. compactus, O. syriaca, O. tytthantum, O. heracleoticum, O. smyrnaeum and O. virens. Further examples of plants from the genus Thymus covered by the term “oregano” are T. herbus-barona, T. citriodorus, T. mastichiana, T. pulegioides. T. serpyllum, T. pallasianus and T. praecox. The concentrate may still contain solvents used for the extraction, be free from them or may be transferred to specific carrier materials. The extracts may be obtained in accordance with methods well-known in the art, e.g., by (an) extraction with solvents like methanol ethanol, ethyl acetate, diethylether, n-hexane, methylene chloride, or with supercritical fluids like carbon dioxide (pure or in mixture with other solvents such as alcohols) or dinitrogen oxide, (b) hydrodistillation for obtaining essential oils or (c) extraction/distillation with hot gases like nitrogen.

Preferably oregano extracts are used that are obtained by an extraction with the use of supercritical carbon dioxide. Such extracts have the advantage that they do not contain any organic solvents, no proteins and no heavy metals. If desired, an extraction with supercritical carbon dioxide is followed by a second supercritical fluid CO2-extraction step to remove waxes and selectively enrich the volatiles.

The oregano extracts or their volatile components can be of natural or synthetic or mixed (viz. partly natural, partly synthetic) origin, i.e., they can, apart from being obtained by extraction of plants and fractionation, be chemically synthesized and, if desired, mixed together in any desired quantities. They can be prepared and used in any desired purities and concentrations, e.g. as solutions containing them in concentrations as low as, e.g., 10% (w/w) or less, or up to nearly 100% (w/w).

Preferred are oregano extracts containing a high proportion of at least one of their volatile components. More preferred are oregano extracts containing at least a total of 70 weight-% of volatile components as mentioned above, based on the total weight of the extract. Completely natural oregano extracts may be fortified with at least one specific volatile component thereof.

Preferred are oregano extracts are oregano extracts which comprise thymoquinone in an amount in the range of at least 2 weight %, and preferably from 2-8 weight % thymoquinone.

Preferred oregano extracts in the context of the present invention are those which also comprise CRV, so that the extract comprises THQ in the range of at least 2 weight %, preferably from 2-8 weight % as above, and also:
at least 30 weight-% of carvacrol, preferably at least 50 weight-% of carvacrol,
more preferably at least 60 weight-% of carvacrol,
and most preferably wherein the oregano extract comprises at least 65 weight-% of carvacrol.

Other preferred oregano extracts are those wherein the oregano extract comprises at least 50 weight-% of carvacrol and from 2 to 25 weight-%, of thymoquinone,
more preferably wherein the oregano extract comprises at least 55 weight-% of carvacrol and at least 2 weight-% of thymoquinone,
even more preferably wherein the oregano extract comprises at least 60 weight-% of carvacrol and at least 2 weight-% of thymoquinone, and
most preferably wherein the oregano extract comprises at least 65 weight-% of carvacrol and thymoquinone in a range of from 2-8 weight-%, based on the weight of the oregano extract.

### The Capsule Shell

The capsule shell can be selected from the group consisting of HPMC, pullulan and gelatin.

However, it has been found in accordance with this invention, that if the capsule shell is either pullulan or gelatin, and the viscosity-increasing agent is phosphatidylcholine or lecithin, then THQ is not stable. Thus, in accordance with this invention, the capsule shell may be:
a) HPMC and may be used with any viscosity-increasing agent;
b) pullulan, with the proviso that the viscosity increasing agent is neither phosphatidylcholine nor lecithin; or
c) gelatin, with the proviso that the viscosity increasing agent is neither phosphatidylcholine nor lecithin.

In a preferred embodiment, the capsule comprises
a) a HPMC shell;
b) oregano extract comprising at least 2 weight % THQ, preferably 2-8% THQ, and further comprising at least 50 weight % CRV, preferably at least 60 weight % CRV;
c) a diluent selected from the group consisting of: middle chain triglycerides, glycerol, edible vegetable waxes; plant oils (for example: olive oil, palm oils, sunflower oils, maize/corn oil, soybean oil, sesame oil, or rice bran oil), and mixtures thereof: and
b) a viscosity-increasing agent.

### The Diluent

The pure oregano extract should not be filled into capsules without mixing it with compatible excipients, since it can damage capsule integrity in high concentration during storage. Suitable diluents include: middle chain triglycerides, preferably oleic acid, edible vegetable waxes; plant oils (for example: olive oil, palm oils, sunflower oils, maize/corn oil, soybean oil, sesame oil, or rice bran oil). The concentration of diluent may vary depending on the particular oregano extract utilized, and as it is sufficient to ensure that the capsule shell remain intact: this can be determined using known methods. It is preferably present at least 50 w/w %, more preferably at least 50-60 w/w %. In some compositions, it is 58 w/w %.

### The Viscosity Increasing Agent

Viscosity modifiers might be needed to adjust the viscosity of the fill for accurate dosing and filling on high speed equipment. Therefore, they are a preferred component of the capsule fill. As the combination of pullulan and phosphatidylcholine or lecithin adversely affect THQ stability, this combination should not be used. Similarly, the combination of gelatin and phosphatidylcholine or lecithin also adversely affect THQ stability, so this combination should not be used. Known viscosity enhancers include: silicum dioxide, stearic acid, cetostearyl, cetyl and stearyl alcohols, glyeryl behenate, glyceryl palmitostearate, several partially or fully hydrogenated glycerides and fatty acid esthers.

In preferred capsules, the viscosity enhancer is silicum dioxide.

It is also preferred that the capsules be opaque or colored to protect from light degradation.

Capsules according to this invention can be assembled in standard ways. Hard capsules containing liquids or semi-solids have to be filled and sealed sequentially in order to prevent leakage. The two commonly used industrial methods for sealing capsules are banding and spray sealing. During banding, a polymer band (e.g. gelatin or HPMC) is used to overlap between the body and cap of the capsule, while a hydroalcoholic solution is sprayed onto the cap's surface to stick the two segments together during spray sealing.

The following non-limiting examples are presented to illustrate the invention.

### EXAMPLES

### Example 1

### 1. Materials and Methods

Oregano extract (OréVida®®, from FLAVEX) was monitored in different compositions during storage in order to study the influence of composition, capsule material and storage conditions on the chemical stability of the THQ and CRV contained in it. Photochemical degradations of THQ were prevented by using opaque/colored capsules. The disintegration time of the capsules was also tested to gain information about possible interactions of THQ or CARV with the capsule shell leading to the formation of a water-insoluble complex, which might result in prolonged capsule disintegration.

### 1.1 Composition of Capsule Fill and Types of Capsule Shells

Since THQ is light-sensitive, the photo degradation was prevented by using opaque/coloured capsules, 00 in size which corresponds to a capsule volume of 0.91 mL. The above-mentioned capsule size and composition 1 (see in Table 1, entry 1) are identical to those of the capsules used in the first human study described in WO 10/094761 (and used for comparison).

Previous studies showed that degradation of THQ could be prevented by diluting the extract or increasing its viscosity. Therefore, silicum dioxide (AEROSIL 200) or phosphatidyl choline (EPIKURON 135 F IP: fractionated soybean lecithin & soybean oil with enriched phosphatidylcholine content) were used as viscosity enhancers. The viscosity of the compositions is approx. 25 mPas at 25° C. without viscosity enhancer, while adding silicum dioxide or phosphatidylcholine increased viscosity up to 52-55 mPas (added amounts included in Table 1).

Middle chain triglycerides (MCT) were utilized as diluents.

TABLE 1
Composition of capsule fills

| | | | | | Quant. | | |
|---|---|---|---|---|---|---|---|
| | | Lab. Cl. | | Ovrg | mg/Capsule | | |
| | | | mg/IU | % | 1 | 2 | 3 |
| 1 | OréVida (Flavex: | 60 | mg | 15 | 69.00 | 69.00 | 69.00 |
| | RV0402-17) | | | | | | |
| 2 | Phosphatidyl- | | | | 231.00 | | |
| | choline | | | | | | |
| 3 | Silicium dioxide | | | | | 4.00 | |
| 5 | Middle Chain | | | | 420.00 | 647.00 | 651.00 |
| | Triglicerides | | | | | | |
| | (MCT) | | | | | | |
| | Total fill weight | | | | 720.00 | 720.00 | 720.00 |

The compatibility of oregano extract was tested in hard gelatin capsules (Coni-Snap®), HPMC capsules (Vcaps®) and pullulan capsules (NPcaps™). Vcaps® and NPcaps™ are declared to be animal-free, preservative-free, gluten-free, non-GMO and GRAS. In addition, both hold Kosher and Halal certificates. All capsule brands were provided by Capsugel (Bornem, Belgium).

The capsules prepared in this study were filled manually by using an Eppendorf micropipette, and capsule banding was done using conventional techniques.

### Example 2

### Capsule Disintegration

Disintegration time was measured by using a DISI-1 disintegration tester (Charles Ischi AG Pharma Prüftechnik, Zuchwill, Switzerland) in 900 ml demineralized water at 37° C. Six parallel measurements were carried out. The upper limit of disintegration time set in USP <2040> is 30 min for hard shell capsules.

### Example 3

### Stability Studies

A long term stability study was performed for 36 months. An accelerated stability study was performed for 6 months at 40° C./75% Relative Humidity (RH). The retention of THQ and CARV was measured and monitored.

The quantification of carvacrol and thymoquinone was done by HPLC-UV. After an extraction with THF/methanol, CARV and THQ are analyzed by RP-HPLC-UV applying a gradient method. The detection wavelengths are set to 254 nm for THQ and 275 nm for CARV. Quantification was carried out by using external standard calibration. The initial assay and content uniformity determination were carried out by analyzing 10 capsules of each batch. Further for the stability, 2 capsules of each batch were analyzed at each time point.

### Stability of THQ and CARV in Liquid-Filled Hard Gelatin Capsules

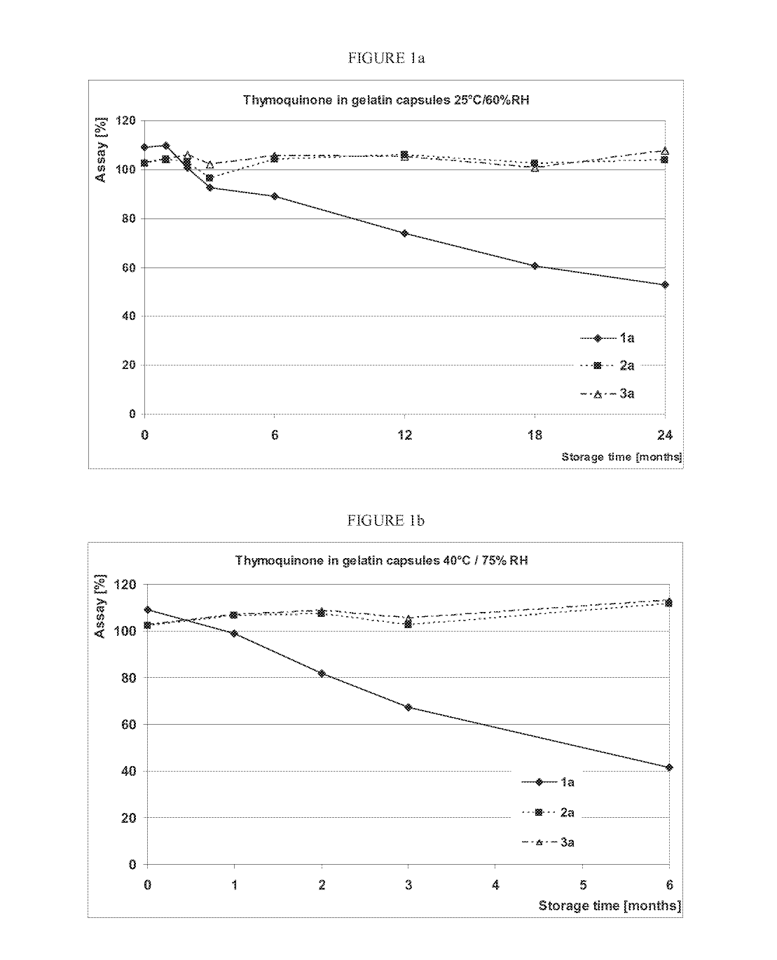

As shown in FIG. 1a and FIG. 1b, THQ shows a good stability in 2 of 3 compositions filled into hard gelatin capsules. A significant decrease in THQ content could already be observed in composition 1 after the first month of storage in the accelerated studies (40° C.). This finding was confirmed by the long-term stability results. As listed in Table 2, capsule formulations 1 contained phosphatidylcholine as a viscosity enhancer.

Our results show that that phosphatidylcholine is incompatible with oregano extract because it promotes chemical degradation of THQ during storage. Since composition 1 was stable during storage at 4° C., it is reasonable to assume that the undesired interaction of THQ with phosphatidylcholine can be prevented at low temperatures.

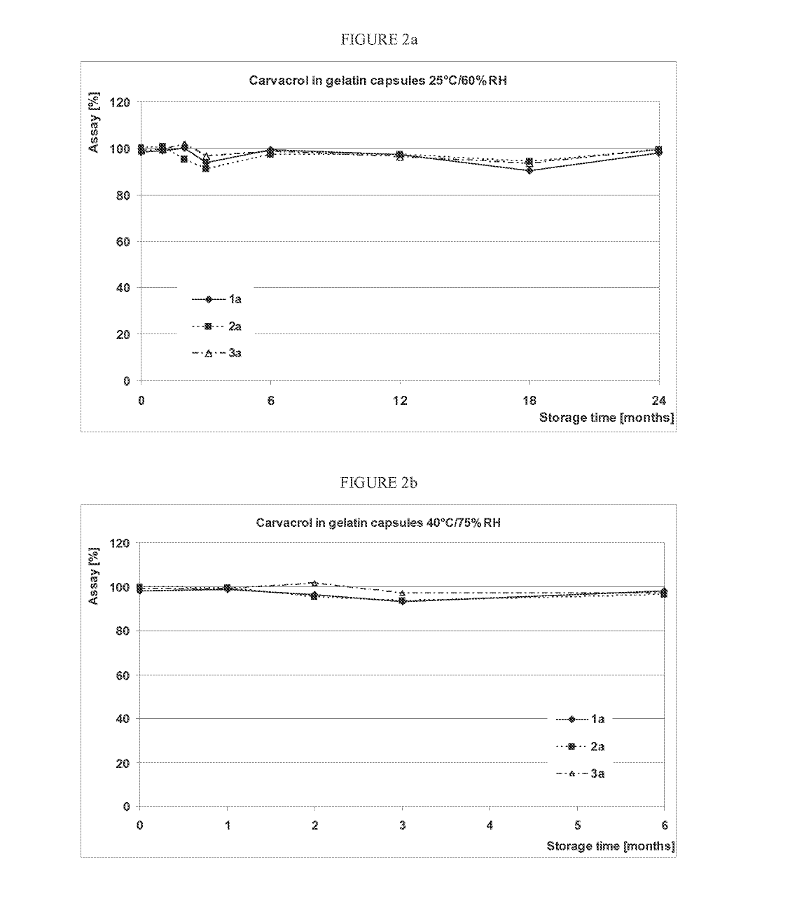

CAR was stable during storage in every capsule formulation (FIG. 2).

TABLE 2
Sample identification in stability studies (these
numbers are referred to in the FIGURES).

| | Capsule | | |
|---|---|---|---|
| | type | Viscosity enhancer | All fill components |
| 1a | Gelatin | Phosphatidylcholine | Orévida, Phosphatidylcholine, MCT |
| 2a | Gelatin | Aerosil | Orévida, Silicium dioxide, MCT |
| 3a | Gelatin | No | Orévida, MCT |
| 1b | HPMC | Phosphatidylcholine | Orévida, Phosphatidylcholine, MCT |
| 2b | HPMC | Aerosil | Orévida, Silicium dioxide, MCT |
| 3b | HPMC | No | Orévida, MCT |
| 1c | Pullulan | Phosphatidylcholine | Orévida, Phosphatidylcholine, MCT |
| 2c | Pullulan | Aerosil | Orévida, Silicium dioxide, MCT |
| 3c | Pullulan | No | Orévida, MCT |

### Stability of THQ and CRV in Liquid-Filled HPMC Capsules

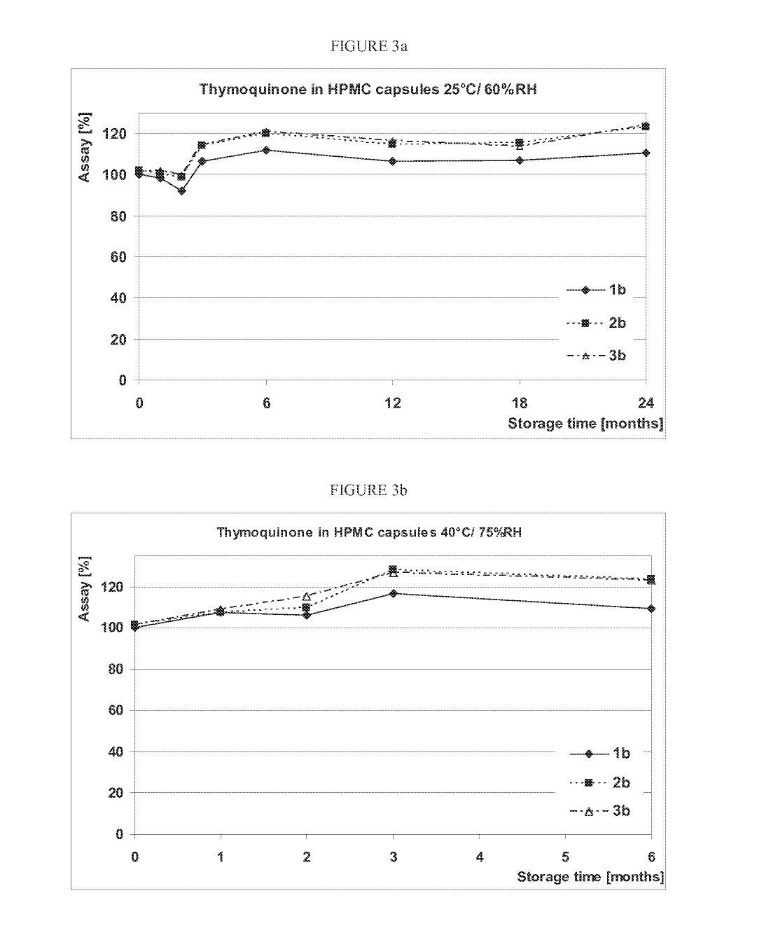

The stability of THQ in HPMC capsules during storage (25° C./60% RH & 40° C./75% RH) is shown in FIG. 3. An increasing THQ content could be measured for the capsule formulations filled into HPMC capsules, which seemed to reach equilibrium after 3 months of storage under accelerated conditions and after 6 months of storage at room temperature (FIG. 3).

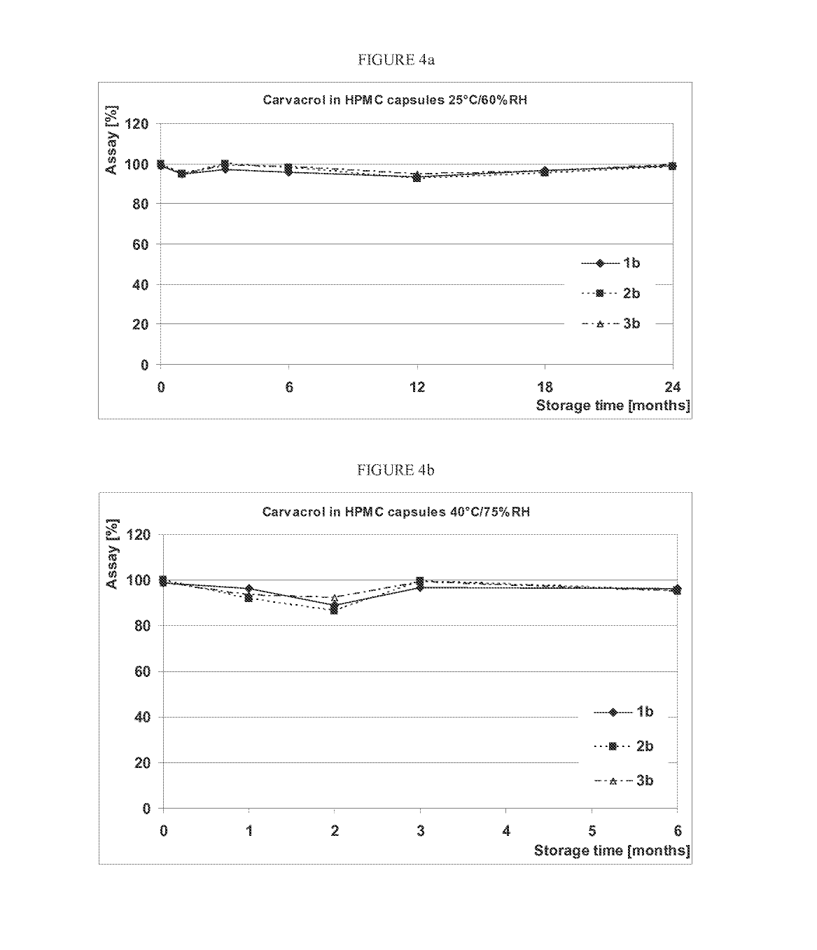

This phenomenon can be explained by the observation that the oxygen permeability of HPMC capsules is higher than those of the gelatin and the pullulan capsules. Since the oregano extract can be regarded as a multi-component mixture, while not wishing to be bound by theory, it appears that a minor component of the extract or small amounts of CRV could be converted to THQ via oxidation during storage. After reaching equilibrium, the chemical degradation of THQ became apparent in composition 1 containing phosphatidylcholine. CRV showed good stability in HPMC capsules (FIG. 4).

### Stability of THQ and CRV in Liquid-Filled Pullulan Capsules

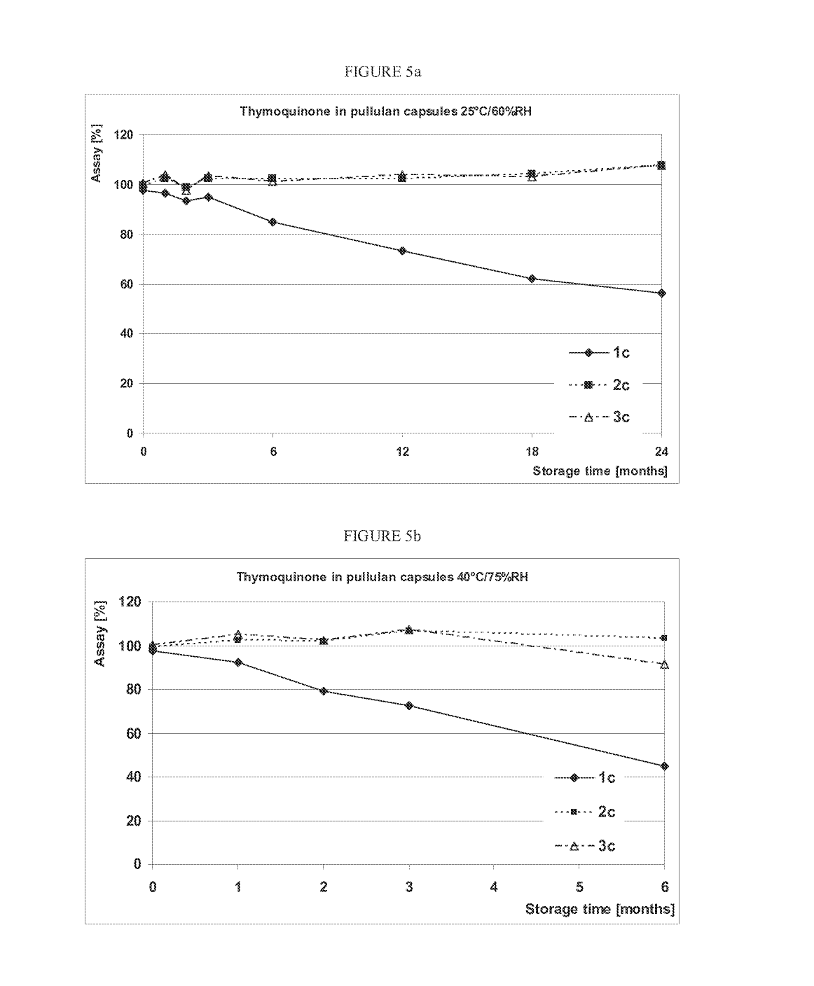

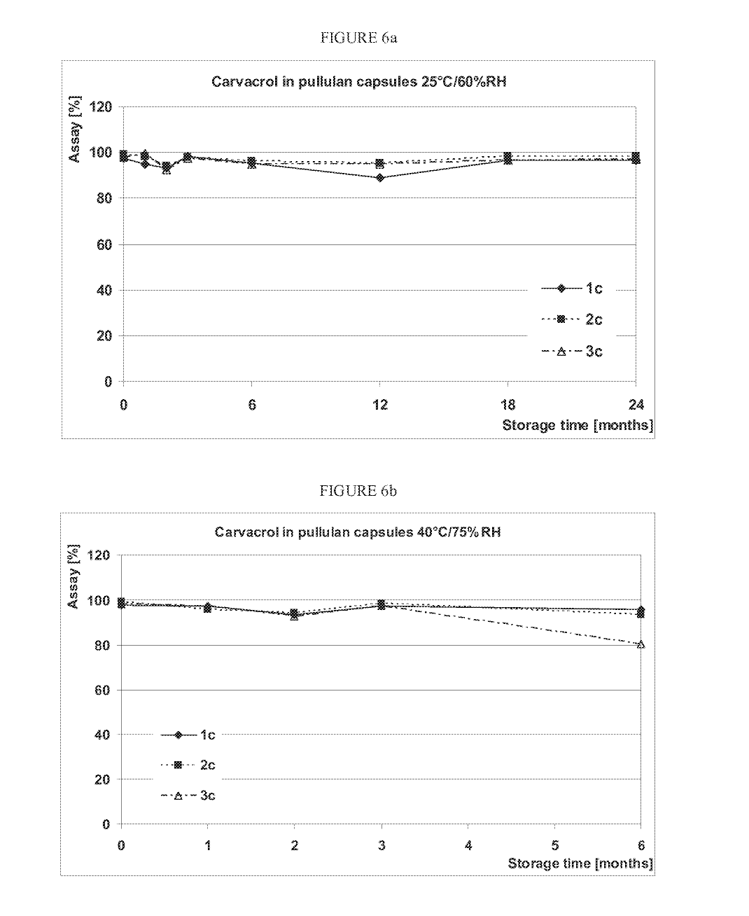

The stability results generated with oréVida oregano extract in pullulan capsules are similar to those observed for gelatin capsules. The capsule composition containing phosphatidylcholine showed inferior stability also in pullulan capsules (FIG. 5). The lower CRV retention measured after 6 months of storage at 40° C. was not confirmed by the long term stability studies. CRV seems to be stable in pullulan capsules as well (FIG. 6).

On the basis of the stability data, it can be concluded that capsule formulations, where oregano extract is mixed and diluted with certain excipients, show good stability in gelatin, HPMC and pullulan capsules, if they are formulated without phosphatidylcholine.

### Example 4

### Disintegration Time

The amino groups of gelatin (lysine residues) tend to cross-link with aldehydes with the passage of time and/or under accelerated stability conditions. Aldehydes are main constituents of essential oils, such as peppermint oil. HPMC is reported to interact with antioxidant polyphenolic actives frequently present in herbal extracts, such as green tea extract. These interactions result in prolonged capsule disintegration and, consequently, retard the rate and extent of dissolution.

In order to test the compatibility of oréVida oregano extract with the capsule shells used in this study, disintegration time of the capsule formulations was measured in 900 ml distilled water at 37° C.

TABLE 3
Disintegration time of the capsules
during storage at 25° C./60% RH

| | Disintegration time (min:s) | | | | |
|---|---|---|---|---|---|
| Sample | 0 | 12 M | 18 M | 24 M | 36 M |

a, gelatin capsules

| | | | | | |
|---|---|---|---|---|---|
| 1a | * | 07:22 | 06:32 | 06:35 | 04:46 |
| 2a | * | 08:06 | 08:12 | 09:48 | 06:33 |
| 3a | 04:55 | 06:21 | 06:05 | 06:53 | 05:38 |

b, HPMC capsules

| | | | | | |
|---|---|---|---|---|---|
| 1b | 06:05 | 05:36 | 08:08 | 05:56 | 05:22 |
| 2b | 06:07 | 05:44 | 07:01 | 06:04 | 04:53 |
| 3b | 05:56 | 05:59 | 06:03 | 05:45 | 04:17 |

c, pullulan capsules

| | | | | | |
|---|---|---|---|---|---|
| 1c | 03:48 | 07:09 | 04:19 | 05:12 | 03:06 |
| 2c | 03:14 | 04:35 | 03:00 | 03:27 | 01:49 |
| 3c | 02:08 | 02:36 | 03:08 | 02:29 | 03:21 |

As shown in Table 3, no significant increase in disintegration time was observed for capsules stored at 25° C./60% RH.

It must be noted, that the oregano extract is strongly diluted in the fill compositions. The concentration of the extract is lower than 10 w/w % in relation to the total fill weight. Diluting the substances, which can potentially react with the capsule shell, might reduce probability of an undesired interaction.

1. A capsule comprising:
a) shell selected from the group consisting of: hydroxypropylmethylcellulose (HPMC), pullulan, and gelatin;
b) a fill composition comprising at least 2 weight % thymoquinone (THQ), preferably from 2-8 weight % thymoquinone;
c) a diluent selected from the group consisting of: middle chain triglycerides, glycerol, edible vegetable waxes; plant oils (for example: olive oil, palm oils, sunflower oils, maize/corn oil, soybean oil, sesame oil, or rice bran oil), and mixtures thereof; and
d) a viscosity increasing agent, with the proviso that the viscosity increasing agent is not phosphaditylcholine, and with the further proviso that when the shell is pullulan or gelatin, then the viscosity increasing agent comprises neither phosphatidylcholine nor lecithin.

2. A capsule according to claim 1 wherein the THQ is stable at room temperature.

3. A capsule according to claim 1 wherein THQ is present in a plant extract.

4. A capsule according to claim 1 wherein the THQ is present in an oregano extract.

5. A capsule according to claim 1 wherein the fill composition further comprises carvacrol (CRV).

6. A capsule according to claim 5 wherein the CRV is at least 30 weight-% of carvacrol, preferably at least 50 weight-% of carvacrol, more preferably at least 60 weight-% of carvacrol, and most preferably least 65 weight-% of carvacrol.

7. A capsule according to claim 1 wherein the shell is HPMC.

8. A capsule according to claim 1 wherein the diluent is middle chain triglycerides.

9. A capsule according to claim 1 wherein the viscosity increasing agent is silicum dioxide.

10. A capsule according to claim 1 wherein the shell is pullulan.

11. A capsule according to claim 1, wherein the shell is gelatin.

12. A capsule according to claim 1 wherein the capsule is opaque.

13. A capsule comprising oregano extract wherein the oregano extract comprises THQ present in an amount of at least 2 weight % when stored at room temperature for the shelf life of the capsule.
